(19) United States
(12) Patent Application Publication
(10) Pub. No.: US 2008/0146868 A1
(43) Pub. Date: Jun. 19, 2008

(54) Adjustable implantable male incontinence device

(76) Inventors: Antoine Jean Henri Robert, Rio de Janeiro (BR); Joao Luiz Schiavini, Rio de Janeiro (BR); Alberto Jose Puscinski, Quilmes (AR)

Correspondence Address: LAURA N. TUNNELL, P.O. BOX 6003, SANTA BARBARA, CA, 93160, US

(21) Appl. No.: 11/582,634

(22) Filed: Oct. 17, 2006

(51) Int. Cl. A61F 2/02

(52) U.S. Cl. 600/31; 600/29

(57) ABSTRACT

An implantable, adjustable male incontinence device. The device has an inflatable balloon affixed to a central portion of a reinforced nonextensible elastomeric strip. The balloon includes a first length of elastomeric tubing extending therefrom that provides fluid communication with the interior chamber of the balloon. The uninflated balloon is implanted to overlie the male urethra with the nonextensible strip oriented horizontally and facing away from the urethra. The lateral opposing ends of the nonextensible strip are sutured to the periosteum of respective left and right iliac bones and the inflation port is implanted beneath the skin in an accessible portion of the body, preferably in the scrotum or, less preferably, in the perineum. The inflation port, which also includes a second length of tubing in fluid communication therewith, is implanted beneath the skin and the first and second lengths of tubing are connected to one another. A fluid is injected into the inflation port and conducted to the interior chamber of the balloon causing it to inflate and force periurethral tissue against the urethra to partially constrict the urethra and reduce the pressure of urine on the external meatus. The urethral constriction can be adjusted by increasing or decreasing the amount of fluid in the balloon via the inflation port.

### BACKGROUND OF THE INVENTION

1. Field of the Invention

The present invention relates to an implantable device operable for adjustably controlling male urinary incontinence.

2. Prior Art

Various implantable devices, such as inflatable medical devices, are known in which the inflatable medical devices are implanted into the tissue of a human to treat urinary incontinence. These devices rely upon constricting the urethra of the patient to maintain continence. For example, Haber et al., in U.S. Pat. No. 4,773,393 discloses an implantable genitourinary prosthesis which provides an inflatable member that is implanted adjacent to the urethra to add bulk to the tissue surrounding the urethra for treating urinary incontinence by increasing tissue volume adjacent the urethra thereby constricting the urethra. U.S. Pat. Nos. 4,802,479 and 4,832,680 to the same inventors disclose istruments for hypodermically dispensing and delivering a fill material to an inflatable balloon implanted adjacent the urethra of a patient for treating urinary incontinence. While the devices of Haber et al. are useful for treating male urinary incontinence, the devices lack means for faciley adjusting the volume of the balloon after implantation. Further, the inflatable member lacks anchoring means operable for preventing postoperative migration of the inflatable member.

U.S. Pat. No. 6,645,138 to Cook et al. discloses an implantable medical device and method for using the device for adjustably constricting a selected body lumen such as a urethra or ureter of a patient to treat urinary incontinence or ureteral reflux. The device includes an adjustable, self-sealing element having a continuous wall, including an inner surface defining a chamber. The adjustable element expands or contracts due to fluid volume introduced into the chamber for restricting a body lumen. Following implantation of the device within a patient, the size of the adjustable element is altered by first locating the adjustable element implanted adjacent the body lumen, and then establishing fluid communication with the adjustable element. The volume of the adjustable element is then adjusted by either introducing or removing volume from the chamber of the adjustable element. While the device may be useful for treating male urinary incontinence, the device lacks means for anchoring the balloon in position such that it doesn't migrate postoperatively. Such postoperative migration can render the device inoperable for exerting upward pressure on the urethra. Further, the device lacks means for easily establishing fluid communication with the balloon to adjust the volume thereof.

U.S. Pat. No. 7,014,606 to Burton et al. discloses an implantable device assembly for controllable coaptation of a body lumen. The implantable device assembly includes an implantable device which includes an adjustable element and a tubular elongate body. The adjustable element includes a continuous wall, including an inner surface defining a chamber. The tubular elongate body includes a peripheral surface, a proximal end and a distal end, where the peripheral surface is connected to and sealed to the adjustable element. The tubular elongate body further includes at least a first interior passageway which extends longitudinally in the tubular elongate body from a first opening at the proximal end to a second opening in fluid communication with the chamber of the implantable device. This allows for adjustably expanding or contracting the adjustable element by applied flowable material introduced through the first opening. The implantable device assembly also includes a sheath, where the sheath includes a wall having an inner surface which defines a channel through which at least a portion of the implantable device can pass. Again, the device lacks anchoring means operable for maintaining the position of the device adjacent the urethra postoperatively and is relatively complicated in construction.

After implantation and inflation, most or all of the forgoing devices maintain pressure on the urethra of the patient and assist with relieving urinary continence. Nevertheless, these devices are prone to being under or over-inflated at the time of implantation. If the devices are over-inflated it may cause the urethra to be over-constricted, and the patient is at risk for retention, a condition wherein the patient cannot pass urine. Such a condition can lead to kidney damage, necessitating major corrective surgery or, at minimum, require the use of a catheter to empty the bladder thereby increasing the risk of urinary tract infection. There remains a need for a male urinary incontinence device that is positively anchored within the patient to prevent migration of the device following implantation and which is readily adjustable postoperatively.

### SUMMARY

The present invention is directed to an implantable male incontinence device and a method for using the device that substantially obviates one or more of the limitations of the related art. To achieve these and other advantages and in accordance with the purpose of the invention, as embodied and broadly described herein, the invention includes an adjustable device adapted for implantation beneath the skin of a male patient for treating urinary incontinence. The device comprises an elongate nonextensible strip having two opposing ends and a central portion disposed midway therebetween. An inflatable elastomeric balloon is affixed to the central portion of the strip. The tissue-contacting surface of the balloon is preferably covered with a layer of open-celled polyurethane foam (not shown) to provide means for further anchoring of the device via tissue ingrowth. The balloon is a flexible elastomeric shell having an interior chamber. The device further includes an inflation port having a fluid reservoir therewithin. A length of tubing provides fluid communication between the interior chamber of the balloon and the fluid reservoir within the inflation port.

The features of the invention believed to be novel are set forth with particularity in the appended claims. However the invention itself, both as to organization and method of operation, together with further objects and advantages thereof may be best understood by reference to the following description taken in conjunction with the accompanying drawings.

### BRIEF DESCRIPTION OF THE DRAWINGS

FIG. 1 is a top view of an adjustable implantable male incontinence device in accordance with the first preferred embodiment of the present invention.

FIG. 2 is a plan anatomical view of a male in a recumbent position illustrating the incontinence device of the present invention implanted within the male to adjustably constrict the urethra, FIG. 2 further illustrating the anchoring of the device by attachment of the nonextensible strip to the iliac bone to prevent postoperative migration of the inflatable member. The strip edges can also be anchored by attaching then to the fibers of the bulbo-spongious muscle around the bulbus or to the aponeurosis fibers that overlie the pelvic muscular diaphragm.

FIG. 3 is a side anatomical plan view of the incontinence device of the present invention implanted within a male patient as illustrated in FIG. 2 and in accordance with the method of the present invention.

### DESCRIPTION OF THE PREFERRED EMBODIMENTS

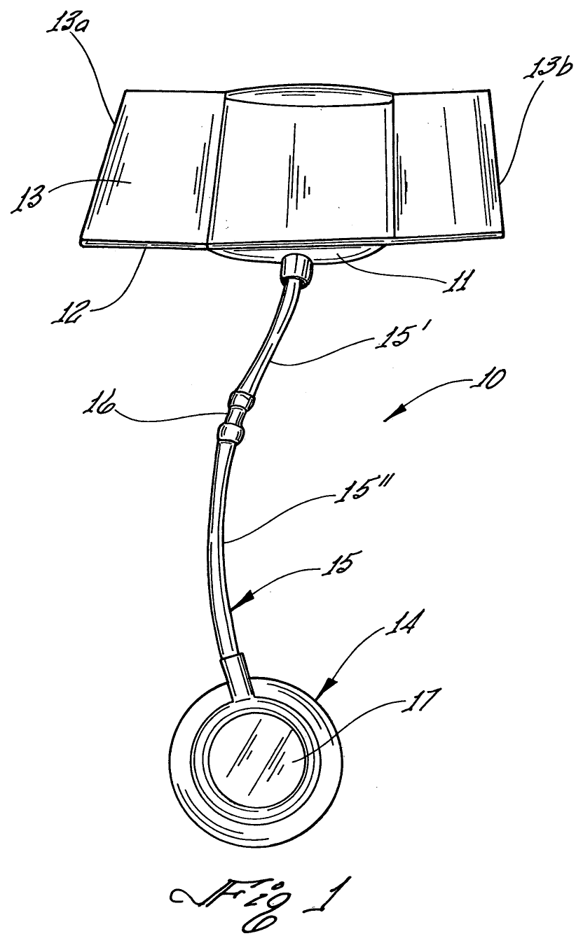

With reference to FIG. 1, an implantable, adjustable male incontinence device of the present invention is indicated at numeral 10. The device 10 comprises an inflatable balloon 11 affixed to a central portion of the lower surface 12 of a nonextensible elastomeric strip 13. The strip 13 is preferably a strip of dacron-reinforced silicone sheeting. Strip 13 has lateral edges 13a and 13b extending laterally from the central portion thereof to which balloon 11 is attached. The balloon 11 has an interior chamber that is in fluid communication with a separable implantable inflation port 14 by tubing 15. The tubing 15 comprises first and second lengths 15′ and 15″ extending from the balloon 11 and the inflation port 14 respectively. The first and second lengths 15′ and 15″ of tubing 15 are, in practice, implanted separately with the balloon and the inflation port respectively, then intraoperatively connected to one another by a tubing connector 16. The inflation port 14 has a chamber therewithin which is sealed by a elastomeric cover 17 which is self-sealing to the track of a hollow-bore needle introduced therethrough for the injection or removal of a fluid into the underlying chamber for adjusting the volume of the balloon 11. It will be understood by the artisan that all parts of the incontinence device 10 that are in contact with human tissue following implantation are made from biocompatible materials.

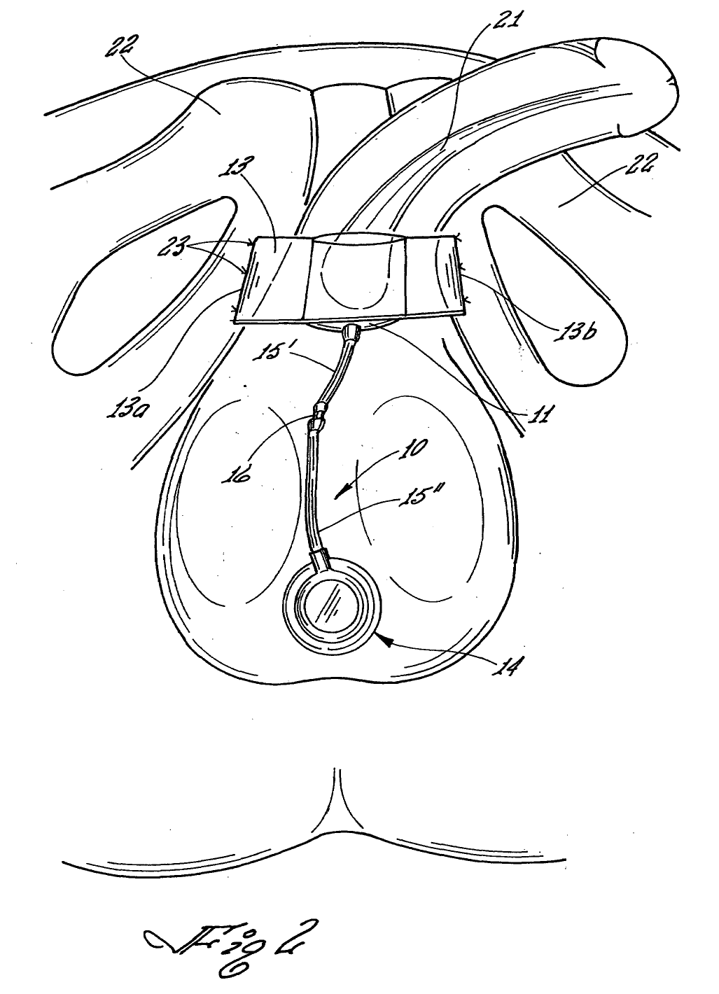

Turning now to FIG. 2, the correct placement of the adjustable urinary incontinence device 10 within the body is illustrated in anatomical perspective view. The balloon 11, which may advantageously include a layer of open-celled polyurethane (not shown) adhered to the tissue-contacting surface thereof, is positioned adjacent to and underlying the urethra 21 with the respective lateral ends 13a and 13b of the nonextensible reinforced strip 13 attached to the iliac bone 22 with sutures 23 to anchor the device 10 and prevent the balloon 11 from migrating from the position shown. The strip 13 and the inflation port 14 are implanted through separate incisions (not shown) in the perineum and the scrotum respectively. The balloon 11 is positioned adjacent to the urethra 21 as shown and the lareral ends 13a and 13b of the strip 13 are sutured to the iliac bone 22. Alternatively, the lateral edges of the strip 13 can also be anchored by attaching then to the fibers of the bulbo-spongious muscle around the bulbus or to the aponeurosis fibers that overlie the pelvic muscular diaphragm.

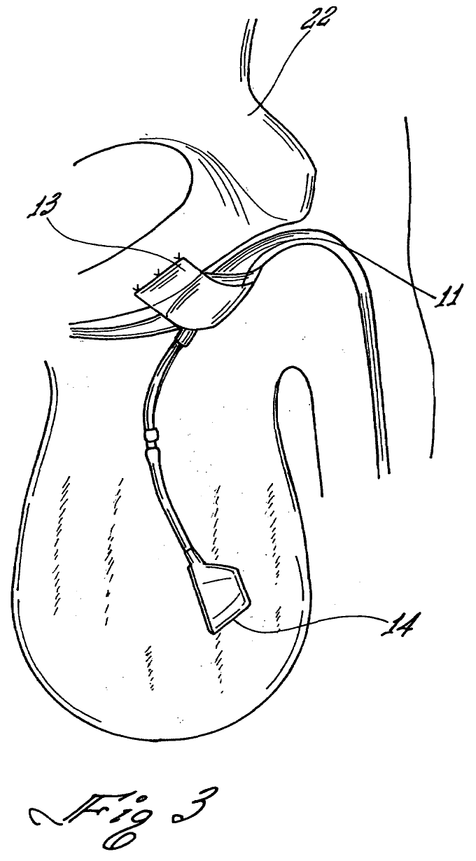

A third incision in the skin is made between the first two incisions and and the tubing portions 15′ and 15″ are extended subdermally to the third incision and connected to one another by tubing connector 16. The incisions are then closed and a fluid such as saline is injected transdermally into the inflation port where it is received and conducted to the interior chamber of balloon 11 by tubing 13. The volume of balloon 11 is adjusted via the inflation port 14 until the pressure bearing on the urethra due to the expansion of the balloon is sufficient to constrict the urethra and prevent the unwanted flow of urine therethrough. A right side anatomical view of the device 10 implanted within the body of a patient, as illustrated in FIG. 2, is shown in FIG. 3. Although it is not included in the drawings, it will be appreciated by those skilled in the art that it is advantageous to adhere a layer of biocompatible foam elastomer such as silicone or, more preferably, open-celled polyurethane foam to the tissue-contacting surface of the balloon to promote tissue ingrowth and provide even more secure anchoring of the balloon.

While particular embodiments of the present invention have been illustrated and described, it would be obvious to those skilled in the art that various other changes and modifications can be made without departing from the spirit and scope of the invention. For example, the inflation port may be implanted in the perineum and still be accessible for transcutaneous introduction of fluids into, or removal of fluid from, the the balloon via a hypodermic needle. It is therefore intended to cover in the appended claims all such changes and modifications that are within the scope of this invention.

What we claim is:

1. An adjustable urinary incontinence device adapted for implantation beneath the skin of a male patient for treating urinary incontinence comprising: (a) an elongate nonextensible strip having two opposing ends and a central portion disposed midway therebetween; (b) an inflatable elastomeric balloon affixed to said central portion of said strip, said balloon having an interior chamber and a first length of tubing extending therefrom in fluid communication with said interior chamber; and (c) an inflation port having a fluid reservoir therewithin and a second length of tubing extending therefrom in fluid communication with said fluid reservoir.

2. The urinary incontinence device in accordance with claim 1 wherein said nonextensible strip has a first length and wherein said central portion of said strip has a second length that is less than said first length.

3. A urinary incontinence device in accordance with claim 2 wherein said nonextensible strip comprises dacron-reinforced silicone elastomer.

4. The urinary incontinence device of claim 3 wherein said inflatable balloon comprises silicone elastomer and wherein said balloon is affixed to said central portion of said strip by vulcanization.

5. The urinary incontinence device of claim 4 wherein said inflatable balloon has a tissue contacting surface and wherein at least a portion of said tissue-contacting surface has a layer of open-celled polyurethane foam adhered thereto.

6. A method for treating urinary incontinence in a male patient comprising the steps of:
(a) presenting a urinary incontinence device in accordance with claim 2; then
(b) making a first incision at the base of the penis and implanting said strip adjacent the urethra such that said balloon underlies the urethra; then
(c) affixing lateral edges of said strip to adjacent portions of the iliac bone; then
(d) making a second incision in the skin and threading said first length of tubing beneath the skin to the second incision; then
(e) making a third incision in the scrotum and implanting said inflation port therewithin and threading said second length of tubing beneath the skin to the second incision; then
(f) connecting adjacent ends of said first and second lengths of tubing to one another; then
(g) injecting a fluid into the inflation port to inflate the balloon; then
(h) closing the first, second and third incisions.

7. The method of claim 6 wherein step (c) is replaced with the step of affixing said lateral edges of said strip to muscle tissue adjacent the iliac bone
